(19) United States
(12) Patent Application Publication
(10) Pub. No.: US 2011/0070663 A1
(43) Pub. Date: Mar. 24, 2011

(54) Method for the enrichment of phosphorylated and/or glycosylated analytes

(75) Inventors: Joerg Von Hagen, Pfungstadt (DE); Uwe Michelsen, Weinheim (DE)

(73) Assignee: Merck Patent Gesellschaft Mit Beschrankter Haftung

(21) Appl. No.: 12/993,116

(22) PCT Filed: Apr. 30, 2009
(86) PCT No.: PCT/EP09/03137

§ 371 Date: Nov. 17, 2010

(30) Foreign Application Priority Data
May 26, 2008 (EP) 08 009 533.4

(51) Int. Cl. C07K 1/22; G01N 33/68

(52) U.S. Cl. 436/501; 530/352; 530/395; 530/344

(57) ABSTRACT

The present invention relates to a method for the purification and isolation of phosphorylated and glycosylated analytes using titanium dioxide particles.

The present invention relates to a method for the purification or isolation of phosphorylated and/or glycosylated analytes using titanium dioxide particles.

Phosphorylation and glycosylation are post-translational modifications which are widespread in nature. It is thought that more than 30% of all human proteins are in phosphorylated form at certain times or under certain conditions.

Phosphorylation and dephosphorylation of proteins plays an essential role in intracellular signal transduction. More accurate data on signal transduction can therefore be obtained by the detection and analysis of phosphorylated proteins. This is of major importance, in particular, for medical questions. For example, the expression of certain phosphorylated or glycosylated proteins in cells could be investigated under certain conditions.

At present, the analysis of phosphorylated or glycosylated proteins is carried out by immunological or mass-spectrometric methods. The poor sensitivity of these techniques requires enrichment methods, which are principally carried out by metal affinity chromatography (IMAC) or antibodies.

EP 1 477 800 discloses the enrichment of phosphorylated analytes using titanium dioxide. M. R. Larsen et al., Molecular & Cellular Proteomics 4, 873-886, 2005, also describe the use of titanium dioxide for the enrichment of phosphorylated peptides. Hitherto, however, the use of titanium dioxide for the enrichment of phosphorylated peptides and proteins has failed because of selective enrichment at the level of the proteins. The existing materials exhibit relatively efficient enrichment at the peptide level, but not at the protein level.

A protein and peptide enrichment method of this type would be of major advantage since it is on the one hand inexpensive and simple to carry out, but in addition also provides information on intact proteins, which are elementary for the understanding of signal transduction analyses, since information on splice variants and further PTMs can also be depicted.

The object of the present invention was therefore to provide an effective method for the enrichment and analysis of phosphorylated and glycosylated analytes, in particular of peptides and proteins.

It has been found that the yield and selectivity of the enrichment can be greatly improved if the phosphorylated and/or glycosylated analytes are enriched with the aid of certain titanium dioxide particles. The titanium dioxide particles according to the invention have a core, preferably comprising magnetite, and an outer surface comprising titanium dioxide, where the outer surface comprising titanium dioxide has not been calcined.

The present invention therefore relates to a method for the enrichment or isolation of glycosylated and/or phosphorylated analytes from a sample, characterised in that the sample is brought into contact with particles, also called titanium dioxide particles below, which have a core to which at least one layer of titanium dioxide which has not been calcined has been applied.

In a preferred embodiment, the surface of the titanium dioxide particles has an irregular shape.

In a preferred embodiment, the sample and the titanium dioxide particles are brought into contact with one another in an acidic binding buffer.

In a preferred embodiment, the titanium dioxide particles are isolated with the bound glycosylated and/or phosphorylated analytes in a subsequent method step, optionally washed, and the analytes are eluted from the titanium dioxide particles using an elution buffer.

In a preferred embodiment, titanium dioxide particles whose titanium dioxide layer has been applied to the core by means of acid precipitation are employed in the method according to the invention.

In a further preferred embodiment, the titanium dioxide particles are in packed form in a column or cartridge.

In a further preferred embodiment, the analytes are phosphorylated proteins and/or peptides. In particular, the method according to the invention exhibits excellent yields in the enrichment or isolation of phosphorylated proteins.

In a preferred embodiment, the core of the titanium dioxide particles consists of magnetite.

In a particularly preferred embodiment, the core of the particles consists of magnetite which has been coated with silicon dioxide.

In a preferred embodiment, the layer of titanium dioxide has been applied to the core by means of acid precipitation.

In a further preferred embodiment, the titanium dioxide particles have a diameter of between 0.025 μm and 50 μm, particularly preferably between 0.1 and 10 μm.

In a preferred embodiment, the proportion by weight of titanium dioxide in the particles is between 20 and 60%.

In a preferred embodiment, the thickness of the titanium dioxide layer is between 3 and 50 nm.

In a preferred embodiment, the layer-thickness difference between the thinnest point of the titanium dioxide coating and the thickest point of the titanium dioxide coating is at least 5 nm, preferably at least 10 nm.

The present invention also relates to a kit, at least comprising titanium dioxide particles which have a core to which at least one layer of titanium dioxide which has not been calcined and preferably has an irregular surface structure has been applied.

In a further preferred embodiment, the titanium dioxide particles are in packed form in a column or cartridge.

In a preferred embodiment, the kit additionally comprises at least one buffer.

In a particularly preferred embodiment, the kit comprises an acidic binding buffer.

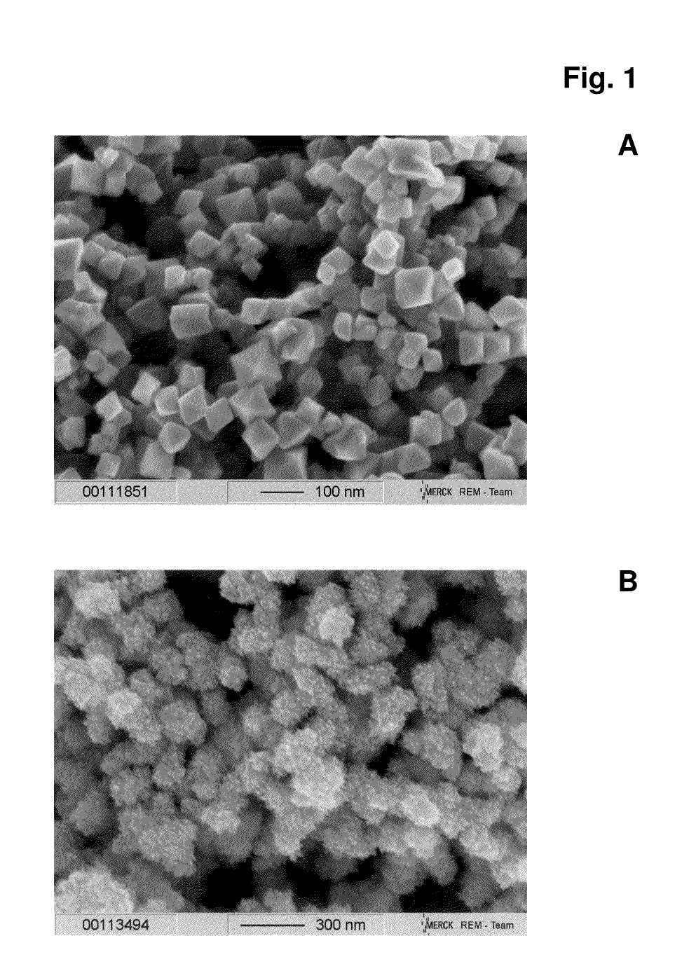

FIG. 1 shows SEM (scanning electron microscope) photomicrographs of the MagPrep® silica particles (A) employed as core and the titanium dioxide particles according to the invention (B).

In accordance with the invention, a sample is any material in which glycosylated and/or phosphorylated analytes, in particular glycosylated and/or phosphorylated peptides and/or proteins, may be present. In particular, these are prokaryotic or eukaryotic cells, cell constituents, body fluids or tissues. The sample may originate from any natural, artificial, genetically engineered or biotechnological source, such as, for example, prokaryotic cell cultures. If the glycosylated and/or phosphorylated analytes are to be purified from cell preparations, the cells are firstly digested by known methods, such as, for example, lysis. If the material to be purified has already been pretreated in another way, lytic digestion can be omitted. Filtration, precipitation or centrifugation steps may be necessary. The person skilled in the art will be able to select a suitable digestion method depending on the source of the sample. In any case, the sample for the method according to the invention should be present in a medium which does not form precipitates or cause other undesired side reactions when the method according to the invention is carried out.

In accordance with the invention, glycosylated and/or phosphorylated analytes are glycosylated and/or phosphorylated molecules or macromolecules, such as, in particular, amino acids, peptides, proteins, sugars or lipids. The glycosylated and/or phosphorylated analytes are particularly preferably glycosylated and/or phosphorylated peptides or proteins.

In accordance with the invention, glycosylated and/or phosphorylated means that the corresponding analytes carry one or more phosphoryl groups and/or carry one or more glycosyl radicals. Preference is given to analytes which have been enzymatically glycosylated and/or phosphorylated. Enzymatic glycosylation is carried out, in particular, by glycosyl transferases (transglycosylation), which catalyse the transfer of activated sugars (usually nucleoside diphosphate sugars), for example, to proteins, peptides or lipids. An example of a natural glycosylation is the attachment of N-acetylglucosamine to the hydroxyl groups of serine or threonine residues.

Proteins are phosphorylated in vivo, for example by protein kinases, where the phosphate esters of L-serine, L-threonine and L-tyrosine residues are formed.

In accordance with the invention, proteins are polypeptides having a molecular weight of at least 5000 Da.

In accordance with the invention, titanium dioxide layer or coating means that the corresponding layer or coating consists for the most part, preferably entirely, of titanium dioxide and/or titanium oxide hydrate (TiO(OH)₂). A layer of this type is preferably formed by acid precipitation of a watersoluble titanium salt in acidic aqueous medium.

In accordance with the invention, titanium dioxide correspondingly means titanium dioxide (TiO₂) and/or titanium oxide hydrate (TiO(OH)₂).

It has been found that certain titanium dioxide particles are particularly effective for the enrichment and extraction of glycosylated and/or phosphorylated analytes. These titanium dioxide particles have a core which has been coated with titanium dioxide. The titanium dioxide coating is characterised in that it does not form a smooth layer on the core, but instead produces an irregular, fissured, raspberry-like structure. This means that the surface of the particles has a large number of roundish, angular or irregularly shaped bumps of different sizes, so that the particle in its outer shape is reminiscent of a raspberry or blackberry or a cauliflower. It is furthermore characteristic of the irregular structure that the titanium dioxide layer on the core is not uniformly thick, but instead only has a layer thickness of one or more molecules in some places. In other places, by contrast, the surface is significantly thicker and has bumps which look as though small titanium dioxide particles have been precipitated onto the core. The titanium dioxide layer is typically at least 5 nm thick at the thinnest point and up to 50 nm at the thickest point. The difference in the layer thickness between the thinnest and thickest points of the coating is at least 5 nm, preferably at least 10 nm. Since there are many points with a rather small layer thickness and many points with a rather large layer thickness, the irregular surface structure, also referred to as fissured or raspberry-like, according to the invention arises.

Without tying oneself to a certain formation mechanism, the characteristic berry structure of the particles according to the invention could arise since small titanium dioxide particles form during the coating of the cores with titanium dioxide and are deposited on the surface, or so-called seed particles, at which further deposition of the titanium dioxide takes place to an increased extent, form on the surface, resulting overall in irregular deposition and therefore in the irregular, raspberry-like structure.

Instead of an irregular, raspberry-like structure, the term agglomerate structure could also be used in the case of the particles according to the invention, since, instead of a smooth film on the surface, a structure forms which looks as though many small titanium dioxide grains have agglomerated on the core. The irregular structure of the particles according to the invention is preferably characterised in that the thickness of the titanium dioxide layer which forms on the core varies on the one hand between 3 and 15 nm (at the thin points, i.e. the notches in the berry structure) and on the other hand between 15 and 40 nm (at the protruding points of the berry structure), and the difference in the layer thickness between the thinnest and thickest points is at least 5 nm, preferably at least 10 nm.

The core of the particles according to the invention can consist of any material which is suitable for coating with titanium dioxide by means of acid precipitation, i.e., in particular, is sufficiently acid-stable. The core here may consist of a uniform material or of an inner core which has been covered with one or more further layers. Examples of suitable core materials are SiO₂, TiO₂, and other metals or metal oxides. The core preferably consists at least partly of a magnetic or magnetisable material, such as, for example, magnetite or maghaemite. The core particularly preferably consists of magnetite, which has been completely or partly coated with SiO₂. The coating of the magnetite with silicon dioxide means that the magnetite cannot be attacked by the acid as easily during the acid precipitation.

An example of a material which is particularly suitable in accordance with the invention for cores comprising magnetite particles whose surface has been coated with silica is MagPrep® silica particles from Merck KGaA, Germany.

The cores employed are preferably monodisperse spherical or irregularly shaped particles having an average diameter of between 0.1 μm and 50 μm, in particular monodisperse particles having a diameter of between 0.5 and 5 μm. In accordance with the invention, monodisperse means that the diameter of the particles varies by less than 10%, preferably less than 50%.

The proportion by weight of the titanium dioxide layer, in particular on use of magnetite cores, is preferably between 20 and 60%.

For the production of the titanium dioxide particles preferably employed in accordance with the invention, the cores are coated with titanium dioxide. The coating is preferably carried out by means of acid precipitation, in which the cores to be coated are initially introduced in aqueous acidic solution. The pH of the aqueous acidic solution is adjusted using conventional acids and caustic lyes, typically using HCl and NaOH. A pH is selected at which the titanium salt employed precipitates out. The pH is typically between 1 and 4, preferably between 1.5 and 3. In the case of titanium oxychloride (TiOCl₂) as titanium salt, the pH is preferably between 1.8 and 2.5, particularly preferably about 2.2.

The acidic suspension which comprises the cores to be coated is then adjusted to temperatures between 40 and 100° C., preferably to temperatures between 65 and 80° C. The salt solution which comprises the dissolved titanium salt is then introduced slowly and uniformly, for example by dropwise addition, into this warmed suspension, typically with stirring or shaking. The content of titanium salt in this salt solution is typically between 0.01 and 5 mol/l. The addition of the salt solution is typically carried out slowly over a number of hours.

Suitable titanium salts besides titanium oxychloride are all titanium salts which are soluble in aqueous solutions and can be precipitated in an acidic pH range. Examples thereof are titanium tetrachloride and titanium sulfate.

It is important that the pH remains stable throughout the reaction, i.e. during the dropwise addition of the salt solution. This can be achieved with the aid of buffer substances (for example phosphate, acetate or citrate buffer) in the reaction solution. However, the addition of further foreign ions is usually undesired, and consequently the adjustment of the pH during the reaction is preferably regulated by parallel addition of acid (for example HCl) or base (for example NaOH, NH₃) as necessary.

When the titanium dioxide layer has reached the desired thickness, the reaction is terminated. This is carried out, for example, by increasing the pH so that the titanium salt no longer precipitates out.

The particles according to the invention obtained in the process are typically filtered off with suction and rinsed. The particles can then remain in the form of a suspension directly until used in aqueous solution. However, they can also be dried by means of vacuum and/or heating to, for example, 100 to 150° C. Conventional processes for the production of titanium dioxide particles are often followed by heat treatment of the particles at temperatures above 500° C.—so-called calcination of the particles. Whereas the titanium dioxide particles immediately after the acid precipitation typically still contain proportions of titanium oxide hydrate or titanium dioxide aquate and at best have nanocrystalline regions, the calcination causes removal of water and the formation of microcrystalline regions. It has been found that the particles according to the invention which have not been calcined, i.e. have never been subjected to temperatures above 500° C., exhibit a much better binding behaviour for phosphorylated and/or glycosylated analytes than corresponding calcined particles.

The particles according to the invention are suitable for all applications in which glycosylated and/or phosphorylated analytes are to be enriched or isolated. In the method according to the invention for the enrichment or isolation of glycosylated and/or phosphorylated analytes, the titanium dioxide particles according to the invention are added to the corresponding sample. This can be carried out at temperatures between 0 and 50° C., preferably between 4 and 20° C., by, for example, adding the titanium dioxide particles in loose form to the sample, incubating them with the sample, for example by careful stirring or shaking, and subsequently separating them off from the sample by sedimentation/centrifugation and decantation, by filtration or the like. In the case of titanium dioxide particles having a magnetic core, the separation can be carried out in a simple manner by applying a magnetic field. Magnetic or magnetisable titanium dioxide particles, in particular, are therefore suitable for automated processes.

The sample can equally be introduced into a cartridge, column, pipette or the like or rinsed through a cartridge, column, pipette or the like which contains the titanium dioxide particles according to the invention, so that the glycosylated and/or phosphorylated analytes are retained on the particles, while the remainder of the sample is eluted.

After the titanium dioxide particles have been separated off from the remainder of the sample, the particles are typically washed with wash buffers.

The elution of the glycosylated and/or phosphorylated analytes is carried out using an elution buffer.

In order to create suitable conditions for binding of the glycosylated and/or phosphorylated analytes to the titanium dioxide particles, the incubation is typically carried out in acidic binding buffers. In accordance with the invention, acidic binding buffers are aqueous buffers which have a pH of below pH 7.5. For the enrichment of peptides and most other analytes, the pH of the acidic binding buffers is preferably between 1 and 3. In the case of proteins, by contrast, a pH of between pH 4 and 7.5, particularly preferably between 6.5 and 7.5, is preferably selected. The acidic binding buffer may comprise, as solvent, water or a mixture of water with up to 90% of a water-miscible solvent. A particularly preferred binding buffer composition comprises water, acetonitrile and TFA (trifluoroacetic acid). In order to prevent non-specific binding of peptides, 0.5 to 5% by weight of DNB (dihydroxybenzoic acid) can also be added to the binding buffer.

The wash buffer employed is typically also an acidic buffer having a pH of less than or equal to pH 7.5. It may have the same or a similar buffer composition to the binding buffer. It is particularly preferred to wash two or more times with different wash buffers. For example, washing is firstly carried out with an acidic, purely aqueous wash buffer and subsequently with an acidic wash buffer which comprises between 10 and 50% of an organic water-miscible solvent, such as, for example, acetonitrile. The acid employed is preferably TFA.

The elution buffers used are generally, in particular in the case of peptides, aqueous, basic buffers, i.e. solutions having a pH of greater than pH 7.5, preferably having a pH of between 8 and 10.5. Suitable bases are, for example, ammonia or NaOH. It is particularly important for the elution buffer to select as far as possible only constituents which do not interfere with the later analytical methods, such as, for example, chromatographic or mass-spectrometric methods. NH₄SCN is preferably also added to the elution buffer.

For the elution of proteins, various elution methods can be selected. On the one hand, the titanium dioxide particles can be taken up, for example, with the bound proteins in the smallest possible amount of a purely aqueous elution buffer having a pH of between 6 and 8, preferably about pH 7.4, which additionally comprises between 0.1 and 2% by vol. of SDS (sodium dodecylsulfate). This mixture is incubated at elevated temperature (50 to 100° C.) and subsequently investigated by gel electrophoresis (for example SDS-PAGE).

On the other hand, proteins can also be eluted using aqueous elution buffers which have a pH of between 6 and 8, preferably about pH 7.4, and additionally comprise a small proportion of a nonionic detergent, such as, for example, between 0.05 and 0.5% by vol. of Triton X 100. After this elution, the proteins can then be sent directly for function assays, such as, for example, enzymatic assays.

Irrespective of the type of analytes, the eluate comprising the glycosylated and/or phosphorylated analytes can be sent for further analyses or applications in various ways. In particular, it can be investigated by liquid chromatography (for example by means of reversed phase chromatography) and/or mass spectrometry. A particularly suitable method is LC-ESI-MS.

The method according to the invention is particularly advantageous if the titanium dioxide particles have a magnetic or magnetisable core. The method can then also be employed for automated enrichment or isolation methods.

Even without further comments, it is assumed that a person skilled in the art will be able to utilise the above description in the broadest scope. The preferred embodiments and examples should therefore merely be regarded as descriptive disclosure which is absolutely not limiting in any way.

The complete disclosure content of all applications, patents and publications mentioned above and below, in particular the corresponding application EP 080095333.4, filed on 26.05.2008, is incorporated into this application by way of reference.

### EXAMPLES

### 1. Production of the Titanium Dioxide Particles

Apparatus: 5 l standard coating apparatus with PLS automation
Amounts used:

| MagPrep ® silica particles | 50 | g |
|---|---|---|
| (Merck KGaA, Germany) | | |
| demineralised water | 950 | g |
| HCl 18% | for the adjustment and | |
| | maintenance of the pH | |
| NaOH 32% | for the adjustment and | |
| | maintenance of the pH | |
| aqueous TiOCl₂ solution | density 1.26 | g/l |
| consumption | 200 | ml |

### Procedure:

A suspension of the MagPrep® silica particles (50 g/l) is heated to 75° C. with stirring. The aqueous TiOCl₂ solution and, if necessary, aqueous HCl or aqueous NaOH solution are then metered in. The aqueous TiOCl₂ solution is metered in at a rate of 0.6 ml/min. The pH during the reaction is pH 2.2.

The particles are subsequently filtered off with suction, rinsed with water and optionally dried in a drying cabinet at 110° C.

FIG. 1 shows SEM (scanning electron microscope) photomicrographs of the MagPrep® silica particles (A) employed as core and the titanium dioxide particles according to the invention (B) produced by the process described above. The irregular surface of the particles according to the invention is clearly evident.

### 2. Binding of SDS Cell Culture Lysates to Titanium Dioxide Particles and Elution with Subsequent Detection by Means of SDS Page and Coomassie Staining

Performance of the experiment:
Wash buffer: 1×PBS
4×SDS sample buffer: 200 mM Tris, pH 6.8
40% of glycerol
8% of SDS
50 mM DTT (freshly added)
   RIPA buffer: 50 mM Tris, pH 7.4
150 mM NaCl
1% (v/v) of Nonidet P-40
0.25% (w/v) of sodium desoxycholate
1 mM EGTA
Before use, add:
1 mM sodium fluoride
1 mM sodium vanadate
1.25 mM phenylmethylsulfonyl fluoride
10 μl/ml of protease inhibitor cocktail III
   Immunoprecipitation wash buffer:
50 mM Tris, pH 7.4
500 mM NaCl
1% (v/v) of Nonidet P-40
0.25% (w/v) of sodium desoxycholate
1 mM EGTA
Before use, add:
1 mM sodium fluoride
1 mM sodium vanadate
1.25 mM phenylmethylsulfonyl fluoride
10 μl/ml of protease inhibitor cocktail III (not for washing the immunoprecipitated sample)

1.8 ml of RIPA buffer are added to 300 μl of MDA MB 468 breast cancer cell lysate (stock solution and supernatant after incubation at time 0. Designation: SL0). In each case, 350 μl of this solution are incubated with the titanium dioxide particles (20 mg). The titanium dioxide particles are rolled overnight at 4° C. The particles are subsequently washed 2× with 300 μl of RIPA each time, subsequently centrifuged off and boiled with 60 μl of SDS sample buffer/DTT. 15 μl thereof are applied to a 10% NUPAGE SDS gel (Invitrogen). The supernatants are mixed in the ratio 2:1 with SDS sample buffer/DTT and boiled at 95° C. for 5 min. 15 μl thereof are applied to the SDS gel.

The titanium dioxide particles employed are on the one hand titanium dioxide particles according to the invention having a particle diameter of about 1 μm produced in accordance with Example 1 and on the other hand conventional particles comprising pure titanium dioxide (Titansphere from GL Sciences, Japan, diameter 5 and 10 μm, surface smooth, calcined).

The titanium dioxide particles according to the invention are significantly more capable of binding proteins (here EGFR and phospho-histone H1) than is the case with the particulate, pure titanium dioxide materials from GL Sciences. In order to confirm that the enrichment mechanism is actually based on the phosphorylation of proteins, Western blot analyses are carried out.

### Western Blot Analysis: Anti EGFR and Anti-Histone H1.

The titanium dioxide particles (10 mg each) are incubated overnight with T×100 cell lysate (diluted 1:4 with RIPA buffer pH 7.4). The particles are subsequently boiled for 10 min with 60 μl of SDS sample buffer in each case comprising 2 mM DTT. The supernatants after the incubation are mixed in the ratio 2:1 with SDS sample buffer and boiled for 10 min, and 15 μl thereof are applied to the gel. The boiled particles are centrifuged. The supernatant (15 μA is applied to the SDS gel.

As evident from the Western blot analyses, 2 phosphorylated proteins are bound to the titanium dioxide particles. Firstly the EGF receptor, which is stimulated in proliferating MDA MB 468 cells, due to the addition of 10% of foetal calf serum, and is thus in phosphorylated form according to the literature. The phosphorylation of histones which is relevant for cell division can likewise be detected.

1. Method for the enrichment or isolation of glycosylated and/or phosphorylated analytes from a sample, characterised in that the sample is brought into contact with particles which have a core to which at least one layer of titanium dioxide which has not been calcined has been applied.

2. Method according to claim 1, characterised in that the sample and the particles are brought into contact with one another in an acidic binding buffer.

3. Method according to claim 1, characterised in that the particles are isolated with the bound glycosylated and/or phosphorylated analytes in a subsequent method step, and the analytes are eluted from the particles usingan elution buffer.

4. Method according to claim 1, characterised in that the particles are in packed form in a column or cartridge.

5. Method according to claim 1, characterised in that the analytes are phosphorylated proteins and/or peptides.

6. Method according to claim 1, characterised in that the layer of titanium dioxide has been applied by means of acid precipitation.

7. Method according to claim 1, characterised in that the core of the particles consists of magnetite which has been coated with silicon dioxide.

8. Method according to claim 1, characterised in that the particles are monodisperse and have a diameter of between 0.1 μm and 10 μm.

9. Method according to claim 1, characterised in that the proportion by weight of titanium dioxide in the particles is between 20 and 60%.

10. Method according to claim 1, characterised in that the thickness of the titanium dioxide layer in the particles is between 3 and 50 nm.

11. Method according to claim 1, characterised in that the layer-thickness difference between the thinnest point of the titanium dioxide coating and the thickest point of the titanium dioxide coating on the particles is at least 5 nm.

12. Kit at least comprising particles which have a core to which at least one layer of titanium dioxide which has not been calcined has been applied.

13. Kit according to claim 12, characterised in that the particles are in packed form in a column or cartridge.

14. Kit according to claim 12, characterised in that the kit additionally comprises at least one buffer.

15. Kit according to claim 12, characterised in that the kit comprises an acidic binding buffer.
